(19) United States
(12) Patent Application Publication
(10) Pub. No.: US 2016/0354033 A1
(43) Pub. Date: Dec. 8, 2016

(54) VITAL SIGN INFORMATION COLLECTION SYSTEM

(71) Applicant: Kabushiki Kaisha Toshiba, Minato-ku (JP)

(72) Inventors: Kazushige OUCHI, Saitama (JP); Isao AMEMIYA, Machida (JP); Takuzo TAKAYAMA, Utsunomiya (JP)

(73) Assignee: Kabushiki Kaisha Toshiba, Minato-ku (JP)

(21) Appl. No.: 15/238,837

(22) Filed: Aug. 17, 2016

Related U.S. Application Data
(63) Continuation of application No. PCT/JP2015/054474, filed on Feb. 18, 2015.

(30) Foreign Application Priority Data
Feb. 19, 2014 (JP) 2014-029592

(51) Int. Cl. A61B 5/00; G06F 19/00; A61B 5/0205

(52) U.S. Cl. CPC A61B 5/681; A61B 5/02055; G06F 19/322; A61B 5/02438

(57) ABSTRACT

A vital sign information collection system has a vital sign information measuring device that measures vital sign information, a vital sign information collecting device that performs authentication by attribute information of a measurement subject and collects the vital sign information measured by the vital sign information measuring device, and links the attribute information with the collected vital sign information, and a management device that acquires the attribute information and the vital sign information linked with each other, from the vital sign information collecting device, and retains the vital sign information linked with personal information to identify the measurement subject.

### CROSS REFERENCE TO RELATED APPLICATIONS

This application is based upon and claims the benefit of priority from the prior Japanese Patent Application No. 2014-29592, filed on Feb. 19, 2014, the entire contents of which are incorporated herein by reference.

### FIELD

An embodiment of the present invention relates to a vital sign information collection system that collects vital sign information.

### BACKGROUND

A system has been proposed that uploads data measured by plural personal devices to a server. In a system of this type, since formats of the data that is transmitted to the server are different from each other between device manufacturers, there is a problem that only the devices manufactured by the same device manufacturer can be incorporated into the system.

In addition, for example, in a case in which vital sign information such as a pulse wave or electrocardiogram is aggregated to the server, it is necessary to thoroughly perform information management so that the vital sign information is not abused.

A vital sign information measuring device that measures vital sign information includes a wearable device that is worn on a body of a person, such as a pulse wave sensor, and a stationary device that is used by many and unspecified persons, such as a scale. Since the stationary device is used by many and unspecified persons, a device is needed that identifies a particular person corresponding to the measurement result, without an error. The wearable device is required to be small and light because of the nature of being worn on a person, and power consumption is also required to be suppressed as much as possible. Therefore, it is difficult to store lots of measurement data in the wearable device, and it is difficult to transmit the measurement data to a distant location in terms of power consumption, even when a wireless transmission function of the measurement data is given to the wearable device.

Thus, an existing system is not capable of centrally managing plural pieces of vital sign information while considering security. An object of the present invention is to provide a vital sign information collection system and vital sign information collecting device capable of centrally managing vital sign information while considering security.

### BRIEF DESCRIPTION OF THE DRAWINGS

FIG. 1 is a diagram illustrating an example of a configuration of a vital sign information collection system 1 according to a first embodiment of the present invention;

FIG. 2 is a block diagram illustrating an example of an internal configuration of a wearable type vital sign information measuring device 2;

FIG. 3 is a diagram illustrating an example of an appearance of a wristband type vital sign information collecting device 3;

FIG. 4 is a block diagram illustrating an example of an internal configuration of the vital sign information collecting device 3;

FIG. 5 is a block diagram illustrating an example of an internal configuration of a server 4;

FIGS. 6A and 6B are block diagrams each illustrating an example of an information terminal 5;

FIG. 7 is a flowchart illustrating an example of a processing procedure according to the first embodiment;

FIG. 8 is a flowchart illustrating an example of a processing procedure of a case in which the vital sign information measuring device 2 ends sensing operation and then the vital sign information collecting device 3 starts sensing operation in a second embodiment of the present invention;

FIGS. 9A and 9B are configuration diagrams illustrating a schematic configuration of a vital sign information collection system 1′ according to a third embodiment of the present invention; and

FIG. 10 is a diagram describing application examples of a vital sign information collecting device according to a fourth embodiment of the present invention.

### DETAILED DESCRIPTION

According to one embodiment, a vital sign information collection system has a vital sign information measuring device that measures vital sign information, a vital sign information collecting device that performs authentication by attribute information of a measurement subject and collects the vital sign information measured by the vital sign information measuring device, and links the attribute information with the collected vital sign information, and a management device that acquires the attribute information and the vital sign information linked with each other, from the vital sign information collecting device, and retains the vital sign information linked with personal information to identify the measurement subject.

Hereinafter, embodiments of the present invention are described with reference to the drawings. Although a configuration and operation in a vital sign information collection system and vital sign information collecting device are mainly described in the following embodiments, a configuration and operation omitted in the following description may exist in the vital sign information collection system and vital sign information collecting device. However, those omitted configuration and operation are included in the scope of the present embodiments.

### First Embodiment

### Vital Sign Information Collection System

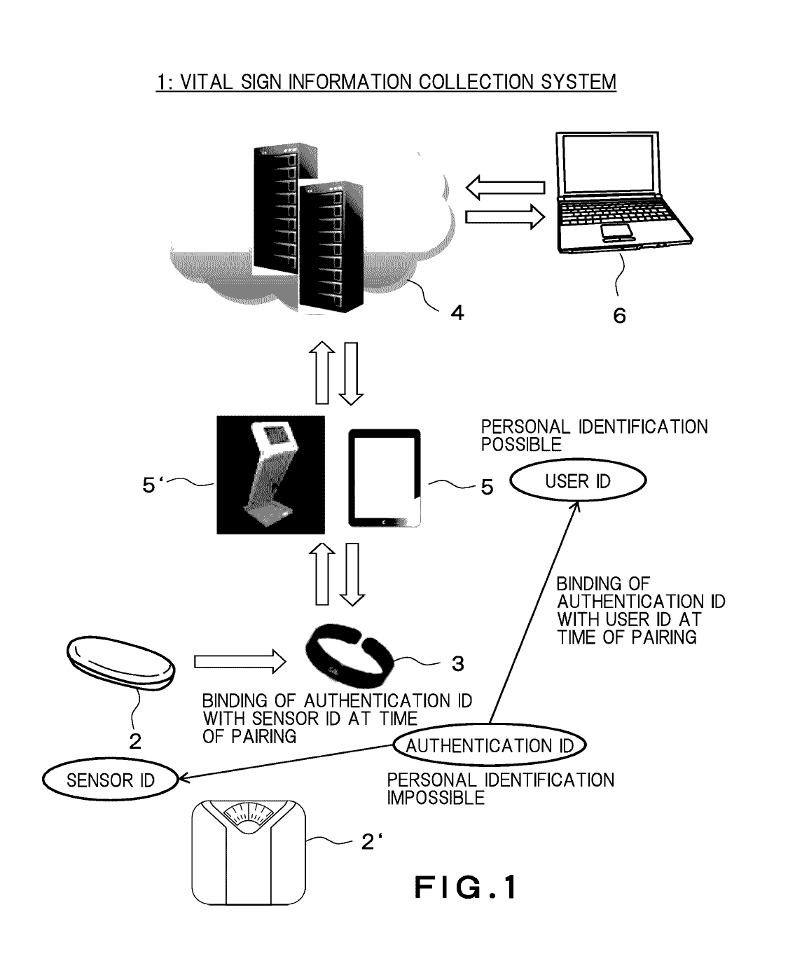

FIG. 1 is a configuration diagram illustrating a schematic configuration of a vital sign information collection system 1. The vital sign information collection system 1 of FIG. 1 includes vital sign information measuring devices 2, 2′ that measure vital sign information, a vital sign information collecting device 3 that performs authentication of a measurement subject and collects the vital sign information measured by the vital sign information measuring devices 2, 2′, and a management device that acquires the vital sign information collected by the vital sign information collecting device 3, and retains the vital sign information and personal information of the measurement subject linked with each other. There is a case in which information terminals 5, 5′ function as the management device, and there is also a case in which the server 4 functions as the management device.

The vital sign information measuring device 2 may be any of a wearable device that is worn on a body of the measurement subject and detects the vital sign information, and a stationary device that is used by many and unspecified persons and detects the vital sign information, such as a scale 2′, or a sphygmomanometer.

Sensing data detected by the wearable device is typically continuous stream type data, and sensing data detected by the stationary device is typically sporadic event type data. In the following, the wearable device is referred to as a wearable type vital sign information measuring device 2, and the stationary device is referred to as a stationary type vital sign information measuring device 2.

The vital sign information collecting device 3 has an authentication ID that is managed by the vital sign information collecting device 3. The authentication ID is attribute information for identifying a measurement subject from other measurement subjects, in the vital sign information collecting device 3. The authentication ID does not necessarily have to be specific information capable of substantially uniquely identifying the measurement subject, such as the name and date of birth, health insurance number, or social security number of the measurement subject. It is rather preferable that the authentication ID is specified information to be capable of identifying the measurement subject in a range in which personal information cannot be identified, in the vital sign information collecting device 3.

The vital sign information collecting device 3, when performing connection setting (pairing) with the vital sign information measuring device 2, retains the ID (sensor ID) of the vital sign information measuring device 2 and the authentication ID of the vital sign information collecting device bound with each other. Various types of vital sign information that are received together with the sensor ID from the vital sign information measuring device 2, and, further, vital sign information collected from plural vital sign information collecting devices 3 are retained linked with the authentication ID of the vital sign information collecting devices 3. With such management, the vital sign information of the measurement subject can be integrally managed in the vital sign information collecting device 3.

The vital sign information collection system 1 of FIG. 1 includes information terminals 5, 5′ that transmit/receive various types of information to/from the vital sign information collecting device 3 and the server 4. The information terminal 5 is, for example, a portable information terminal such as a PC, smart phone, or PDA. The information terminal 5′ is, for example, a kiosk type information terminal that includes a display screen and an input unit, which is fixedly installed in public agencies such as streets and shops, hospitals, nursing facilities, and sports facilities.

When the measurement subject logs in to the information terminals 5, 5′, and vital sign information is transmitted from the vital sign information collecting device 3 to the information terminals 5, 5′, it is possible to retain the vital sign information linked with login information in the information terminals 5, 5′. Thus, the vital sign information measured by the vital sign information measuring device 2 can be integrally managed by the information terminals 5, 5′ together with the login information.

Here, by associating the login information with the user ID, it becomes possible to retain the user ID, authentication ID, and vital sign information linked with each other in the information terminals 5, 5′. The user ID is preferably the information capable of uniquely identifying the measurement subject, such as the name and date of birth, mail address, health insurance number, or social security number.

The vital sign information collection system 1 of FIG. 1 may include an information browsing terminal 6 that browses a part of the vital sign information stored in the server 4. For example, the information browsing terminal 6 acquires from the server 4 the vital sign information about the measurement subject to which authentication has been performed, and analysis data analyzed by the server 4 in relation to the vital sign information, and displays the information and the data. Incidentally, the function of the information browsing terminal 6 may be included in the information terminals 5, 5′. That is, the information terminals 5, 5′ may each have a function of transmitting the vital sign information to the server 4, and a function of acquiring necessary information from the server 4 and displaying the information.

Incidentally, in the present embodiment, the information browsing terminal 6 is not essential for the vital sign information collection system 1, and is provided if necessary.

The vital sign information collecting device 3, as described later, is carried by a user, and memory capacity of the device is limited. Therefore, the vital sign information collecting device 3 may delete the vital sign information when transmission of vital sign information to the server 4 or information terminals 5, 5′ is completed.

<Vital Sign Information Measuring Device>

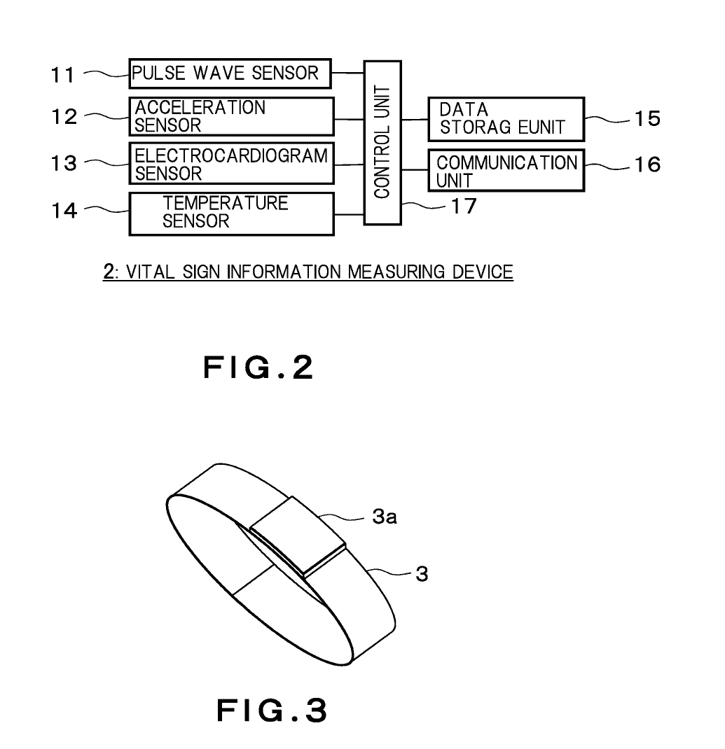

FIG. 2 is a block diagram illustrating an example of an internal configuration of the wearable type vital sign information measuring device 2. The vital sign information measuring device 2 of FIG. 2 includes a pulse wave sensor 11 that measures a pulse wave, an acceleration sensor 12 that detects motion of the body, an electrocardiogram sensor 13 that measures a heartbeat; a temperature sensor 14 that measures body temperature, a data storage unit 15 that stores sensing data of those sensors, a communication unit 16 that communicates with the vital sign information collecting device 3, and a control unit 17 that performs control of an entire of an inside of the vital sign information measuring device 2.

The vital sign information measuring device 2 of FIG. 2 is capable of analyzing, for example, a sleep state of a person by using vital sign information detected by the pulse wave sensor 11, acceleration sensor 12, and electrocardiogram sensor 13. More specifically, a sleep state and an awake state are identified from the motion of the body detected by the acceleration sensor 12, and fluctuation of the heartbeat or pulse is acquired by the electrocardiogram sensor 13 or pulse wave sensor 11, and sympathetic nerve activity and parasympathetic nerve activity are obtained by frequency analysis of the fluctuation of the heartbeat or pulse, and the sleep state is determined from balance of these activities.

In addition, the vital sign information measuring device 2 of FIG. 2 is also capable of analyzing the body temperature and the motion of the body, so that the vital sign information measuring device 2 can be utilized for sleep management, exercise management, and health management. The analysis of the sleep state described above, or the like may be performed by using the measurement data of each sensor in the vital sign information measuring device 2; alternatively, only sensing may be performed in the vital sign information measuring device 2, and the analysis of the measurement data, or the like may be performed by the vital sign information collecting device 3, information terminals 5, 5′, or server 4.

Incidentally, a type of the sensor provided in the wearable type vital sign information measuring device 2 is not limited to the sensor illustrated in FIG. 2. For example, a sensor that measures blood oxygen saturation level, a sensor that measures blood pressure, and a sensor that measures brain waves may be provided.

Since the data storage unit 15 is provided in the vital sign information measuring device 2 of FIG. 2, the vital sign information detected by each sensor can be once stored in the data storage unit 15, and moved from the data storage unit 15 to the communication unit 16 at a predetermined timing, and transmitted to the vital sign information collecting device 3. Incidentally, it is not essential to provide the data storage unit 15 in the vital sign information measuring device 2, and the vital sign information detected by each sensor may be transmitted at any time to the vital sign information collecting device 3 without being stored.

Some of the sensors in the vital sign information measuring device 2 of FIG. 2 is made to adhere with adhesive material such as an adhesive bandage or to be in close proximity sandwiching a small space to skin of a person, and each sensor is capable of performing continuous sensing while adhering or being in close proximity to the skin of the person.

Incidentally, the number of the vital sign information measuring devices 2 is not limited to one, and the plural vital sign information measuring devices 2 that respectively measure different kinds of vital sign information may be provided in the vital sign information collection system 1. The vital sign information measuring device 2 is not limited to the device incorporating the plural sensors illustrated in FIG. 2, and plural vital sign information measuring devices 2 each incorporating a single or a small number of sensors may be provided in the vital sign information collection system 1.

<Vital Sign Information Collecting Device>

The vital sign information collecting device 3 is a sensor hub capable of collecting the data measured in the vital sign information measuring device 2. The vital sign information collecting device 3 is possessed by the measurement subject whose vital sign information is measured by the vital sign information measuring device 2, and is considered to be made into a wristband type that is worn on an arm of a person, as illustrated in FIG. 3, for example. The vital sign information collecting device 3 may be provided with a function unit 3a to have a function of performing authentication by the vital sign information such as a vein or fingerprint by contacting a finger, a display function of displaying the vital sign information acquired and an analysis result of the vital sign information, or the like. A wearing form of the measurement subject can be variously modified to, for example, a ring type, adhesive bandage type, or eyeglasses wearable type.

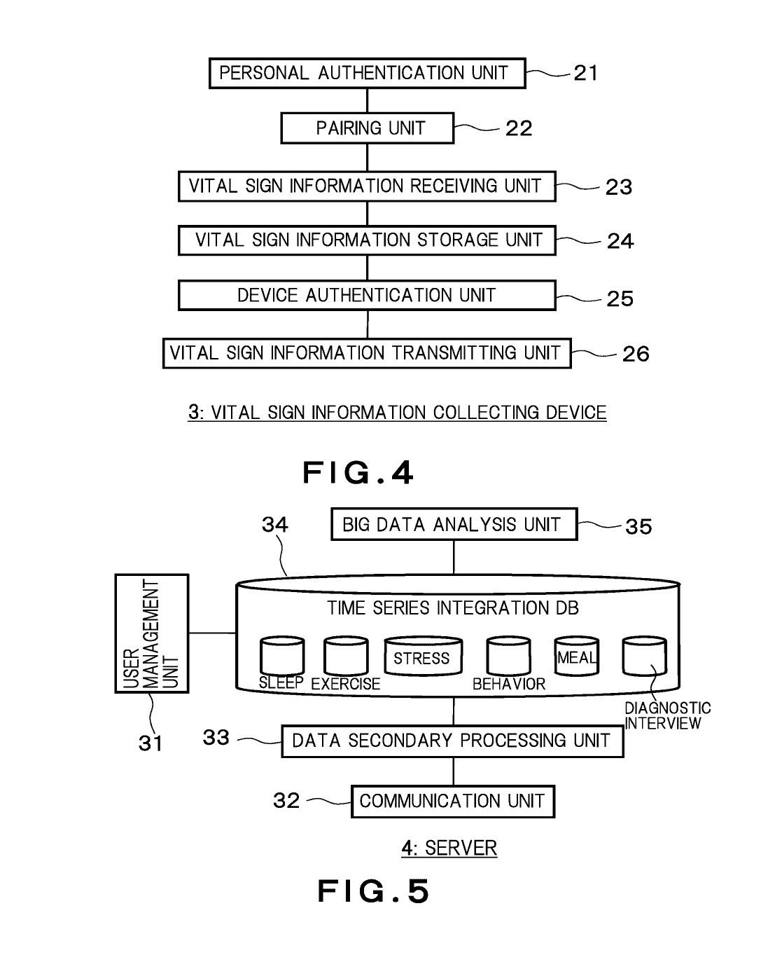

FIG. 4 is a block diagram illustrating an example of an internal configuration of the vital sign information collecting device 3. The vital sign information collecting device 3 of FIG. 4 includes a measurement subject authentication unit 21, a pairing unit 22, a vital sign information receiving unit 23, a vital sign information storage unit 24, a device authentication unit 25, and a vital sign information transmitting unit 26.

The measurement subject authentication unit 21 authenticates the measurement subject who possesses the vital sign information collecting device 3. For a specific method of authentication, various techniques can be applied, such as authentication by using the vital sign information such as a retina, vein, or fingerprint, authentication by a voice print, or authentication by input information such as a password.

A function of detecting attachment and detachment of the vital sign information collecting device 3 may be provided to start authentication processing by the measurement subject authentication unit 21 automatically when the vital sign information collecting device 3 is worn on the measurement subject. In this way, usability for the user in the authentication processing can be improved. In addition, authentication information at the time of authentication may be validated while the vital sign information collecting device 3 is worn on the person after the automatic or manual authentication processing, and the authentication information may be invalidated when the vital sign information collecting device 3 is taken off. Thus, high level of security can be achieved.

The pairing unit 22 pairs the vital sign information collecting device 3 and the vital sign information measuring device 2 with each other, and performs linking of the measurement subject who possesses the vital sign information collecting device 3 and the vital sign information measured by the vital sign information measuring device 2. In a case in which the wearable type vital sign information measuring device 2 is used, pairing processing is preferably performed between the vital sign information measuring device 2 and the vital sign information collecting device 3 before starting measurement of the vital sign information by the vital sign information measuring device 2. As for a specific technique of the pairing processing, it is considered to perform linking of the vital sign information measuring device 2 that is used, for each measurement subject authenticated by the vital sign information collecting device 3. At the time of the linking, the sensor ID of the vital sign information measuring device 2 and the authentication ID of the vital sign information collecting device 3 are used.

In this case, in a case in which the vital sign information collecting device 3 is taken off from the body of the person, the vital sign information collecting device 3 preferably be automatically disabled to acquire the vital sign information from the vital sign information measuring device 2. Thus, abuse of the vital sign information can be prevented, and the vital sign information of a different person is no longer collected erroneously, so that reliability of the vital sign information collected by the vital sign information collecting device 3 can be improved.

It is considered that the pairing unit 22 performs pairing processing between the vital sign information collecting device 3 and the stationary type vital sign information measuring device 2 such as the scale 2′ or the sphygmomanometer, with any of the following procedures of (1) to (5).

(1) Device authentication is performed in advance between the vital sign information measuring device 2 and the vital sign information collecting device 3, and after the measurement by the vital sign information measuring device 2, the vital sign information is transmitted from the vital sign information measuring device 2 to the vital sign information collecting device 3.

(2) The vital sign information is measured by the vital sign information measuring device 2, and then the vital sign information collecting device 3 is brought close to the vital sign information measuring device 2 to acquire the vital sign information. In this case, it is assumed that the vital sign information collecting device 3 and the vital sign information measuring device 2 each have a short-range wireless communication function such as NFC (Near Field Communication: registered trademark), or Bluetooth (registered trademark).

(3) It is made so that the vital sign information is measured by the vital sign information measuring device 2, and then a list of communication devices with which the vital sign information collecting device 3 can communicate is displayed on a display screen of the vital sign information collecting device 3, and when the user selects a specific vital sign information measuring device 2 from the list, the vital sign information can be acquired from the vital sign information measuring device 2. In this case, it is assumed that the vital sign information collecting device 3 includes a display function and an information selection function.

(4) The vital sign information is measured by the vital sign information measuring device 2, and then the vital sign information measuring device 2 automatically transmits the vital sign information to a vital sign information collecting device 3 in the closest location.

(5) Before the measurement of the vital sign information is started by the vital sign information measuring device 2, the vital sign information measuring device 2 and the vital sign information collecting device 3 are in contact with each other, or arranged in close proximity to perform authentication, and the vital sign information measured by the vital sign information measuring device 2 is automatically acquired by the vital sign information collecting device 3 with which authentication has been performed.

The vital sign information receiving unit 23 in the vital sign information collecting device 3 of FIG. 4 receives the vital sign information transmitted from the vital sign information measuring device 2 to which the pairing is performed with any procedure described above, and stores the vital sign information in the vital sign information storage unit 24. Transmission and reception of the vital sign information between the vital sign information collecting device 3 and the vital sign information measuring device 2 are wirelessly performed. A specific form of the wireless does not matter, and, for example, Bluetooth (registered trademark), infrared communication, other various types of short-range wireless communication, and wireless LAN are considered.

The device authentication unit 25 performs authentication with the information terminal 5. The vital sign information transmitting unit 26 transmits the vital sign information collected by the vital sign information collecting device 3 together with the authentication ID to the information terminal 5 to which device authentication has been performed.

As described in a third embodiment, in a case in which the vital sign information collecting device 3 performs transmission/reception of the data directly to/from the server 4, the device authentication unit 25 performs device authentication with the server 4. In this case, the vital sign information transmitting unit 26 transmits the vital sign information collected by the vital sign information collecting device 3 to the server 4 to which device authentication has been performed. Incidentally, after the user logs in from the server 4 or the information terminals 5, 5′ with the ID and password, it is also possible to manually register the vital sign information collecting device 3 of the user, and it is possible to omit the device authentication unit 25 in this case.

The vital sign information transmitting unit 26 transmits the authentication ID, the vital sign information, and the like to the server 4 and information terminal 5. At this time, information granularity may be adjusted of the vital sign information that is transmitted to the server 4 and the information terminal 5. For example, the vital sign information collecting device 3 may determine whether or not there is abnormality in the vital sign information, and transmit the vital sign information appropriately thinned out in a case in which it is determined that the vital sign information is normal, and transmit detailed vital sign information in a case in which it is determined that there is abnormality. Specifically, the vital sign information collecting device 3 has a function of retaining raw data for the latest predetermined time, and deletes the data at any time in a case in which the vital sign information does not have abnormality. In a case in which it is determined that the vital sign information has abnormality (for example, arrhythmia), the raw data (electrocardiogram or pulse wave) around the abnormality is transmitted. The data around the abnormality is, for example, the data that is retained at the time when it is determined that there is abnormality, and the data from that time until when it is determined that the abnormality is ended.

<Management Server>

FIG. 5 is a block diagram illustrating an example of an internal configuration of the server 4. The server 4 of FIG. 5 includes a user management unit 31, a communication unit 32, a data secondary processing unit 33, a time series integration database 34 (herein after, time series integration DB), and a big data analysis unit 35.

The user management unit 31 manages authentication information and personal information of the user. The communication unit 32 acquires the vital sign information and the like from the information terminals 5, 5′ in a case in which the information terminals 5, 5′ exist, and acquires the vital sign information and the like from the vital sign information collecting device 3 in a case in which the information terminals 5, 5′ do not exist. The data secondary processing unit 33 performs processing of classifying the vital sign information acquired, for example, for each type and each user, processing of analyzing the vital sign information, and the like.

The time series integration DB 34, for example, finely divides and stores the vital sign information. In FIG. 5, an example is illustrated in which the vital sign information is divided into a sleep, exercise, stress, behavior, meal, diagnostic interview to be stored; however, the vital sign information is not limited thereto.

As time elapses, a large amount of data is stored in the time series integration DB 34, and the data becomes big data eventually. The big data includes the vital sign information over a long period of a large number of users, and is valuable data that can be used for various analyses. For example, for each age or gender, a sleep tendency or behavior pattern can be analyzed, and health management can be performed. Therefore, for example, various analyses can be performed by providing the big data analysis unit 35 in the server 4, or outside the server 4. In some cases, big data may be generated including the vital sign information from which the user information is deleted, and the data may be sold. Incidentally, the big data analysis unit 35 is not an essential configuration unit of the present system.

FIG. 5 is an example of the server 4, and the internal configuration of the server 4 is not limited to the configuration illustrated in FIG. 5. In addition, the plural servers 4 may be provided. In addition, a separate server 4 may be provided for each type of the vital sign information, or for a specific user group. Further, it may be made so that the vital sign information and the like are transmitted to the server 4 from the information terminal 5 and the vital sign information collecting device 3, and, when the server 4 completes reception of the vital sign information and the like, the information indicating the completion is transmitted from the server 4 to the information terminal 5 and the vital sign information collecting device 3, and the information terminal 5 and the vital sign information collecting device 3 that receive the information delete the transmitted vital sign information. Thus, overflow of the memory capacity can be prevented in the information terminal 5 and the vital sign information collecting device 3. The vital sign information stored in the vital sign information measuring device 2 may also be deleted at the timing of deletion of the vital sign information stored in the information terminal 5 and the vital sign information collecting device 3.

<Information Terminal>

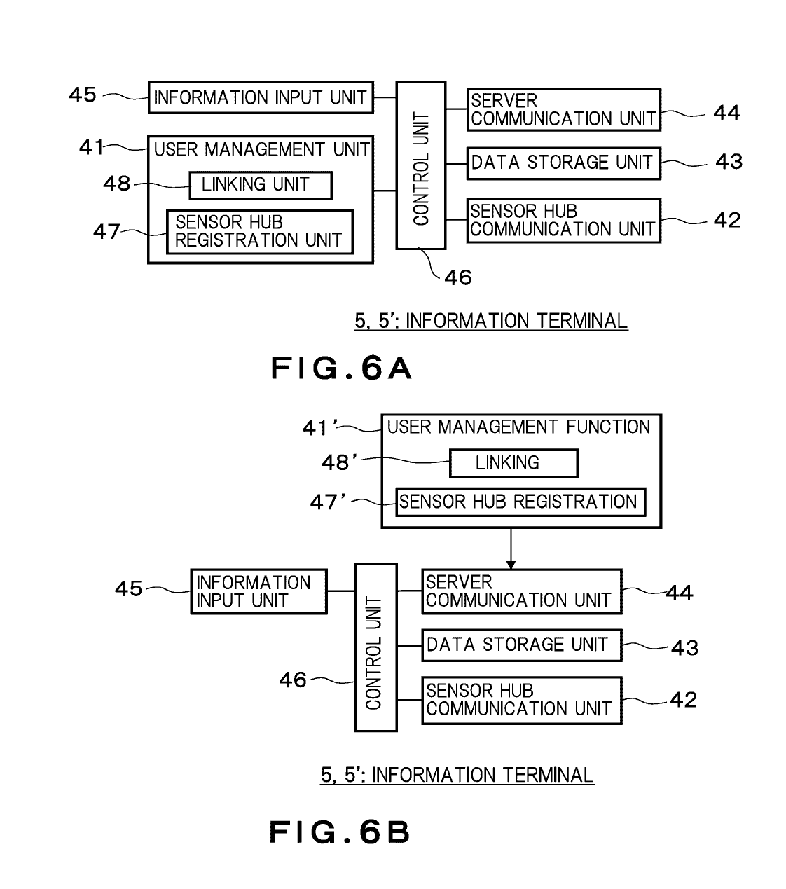

FIGS. 6A and 6B are block diagrams illustrating examples of information terminals 5, 5′. The information terminals 5, 5′ of FIG. 6A each include a user management unit 41, a sensor hub communication unit 42, a data storage unit 43, a server communication unit 44, an information input unit 45, and a control unit 46. The information terminal 5 that includes the user management unit 41 is a management device that retains the vital sign information linked with the personal information capable of identifying the measurement subject.

The user management unit 41 includes a sensor hub registration unit 47, and a linking unit 48. The sensor hub registration unit 47 registers identification information of the vital sign information collecting device 3 that is an object that acquires the vital sign information. The linking unit 48 links a measurement subject who logs in using the information terminal and the vital sign information collecting device 3 that possesses the vital sign information of the measurement subject with each other. The linking unit 48, for example, performs authentication processing between the information terminal 5 and the vital sign information collecting device 3 after the measurement subject has logged in to the information terminal 5, and, when the vital sign information collecting device 3 possesses the information of the user who has logged in, links the vital sign information collecting device 3 with the user who has logged in.

The login to the information terminal 5 and the login to the server may be performed separately. For example, as illustrated in FIG. 6B, a user management function 41′ may be prepared in a cloud server side, and a service login screen may be deployed in the information terminals 5, 5′ with the user management function 41′. In this case, the ID and password are registered with the cloud server, and login to the server is performed by accessing from a browser. That is, the server 4 becomes the management device that retains the vital sign information linked with the personal information capable of identifying the measurement subject.

In authentication of the vital sign information collecting device 3 by the measurement subject authentication unit 21, authentication information from a fingerprint or the like and a unique ID (authentication ID) are managed associated with each other, and the sensing data can be bound with an individual by associating the user management by the user management function 41′ with the authentication ID.

In this way, in the authentication of the vital sign information collecting device 3 by the measurement subject authentication unit 21, management is performed in a state in which the individual cannot be uniquely identified. On the other hand, in the user management by the user management unit 41, user management function 41′, the individual can be substantially uniquely identified. It becomes possible to perform management with higher level of security by integrally managing these in the server 4 side.

In FIGS. 6A and 6B, the sensor hub communication unit 42 receives the vital sign information wirelessly transmitted from the vital sign information collecting device 3 through Bluetooth, other short-range wireless communication, or the like, and stores the vital sign information received in the data storage unit 43.

The server communication unit 44 transmits the vital sign information stored in the data storage unit 43 to the server 4.

In a case in which the information terminal 5 has an advanced data processing function, the vital sign information stored in the data storage unit 43 may be analyzed, and distinctive vital sign information may be extracted and transmitted to the server 4. Thus, communication traffic with the server 4 can be reduced, and processing can be simplified in the data secondary processing unit 33 in the server 4.

The information input unit 45 is an input device such as a keyboard, and inputs various types of information.

Various types of related information (correct data) about the vital sign information of the user may be input by using the information input unit 45. For example, information about a detail of eat and drink, taking medicine, physical condition, exertion, and the like may be input by the user with the information input unit 45. In order to help information input of the user, information of the diagnostic interview may be displayed on a display screen of the information terminal 5, and the user may answer the diagnostic interview via the information input unit 45. By inputting the correct data, a detailed analysis is possible by looking back what the user has done at the time, about the data measured.

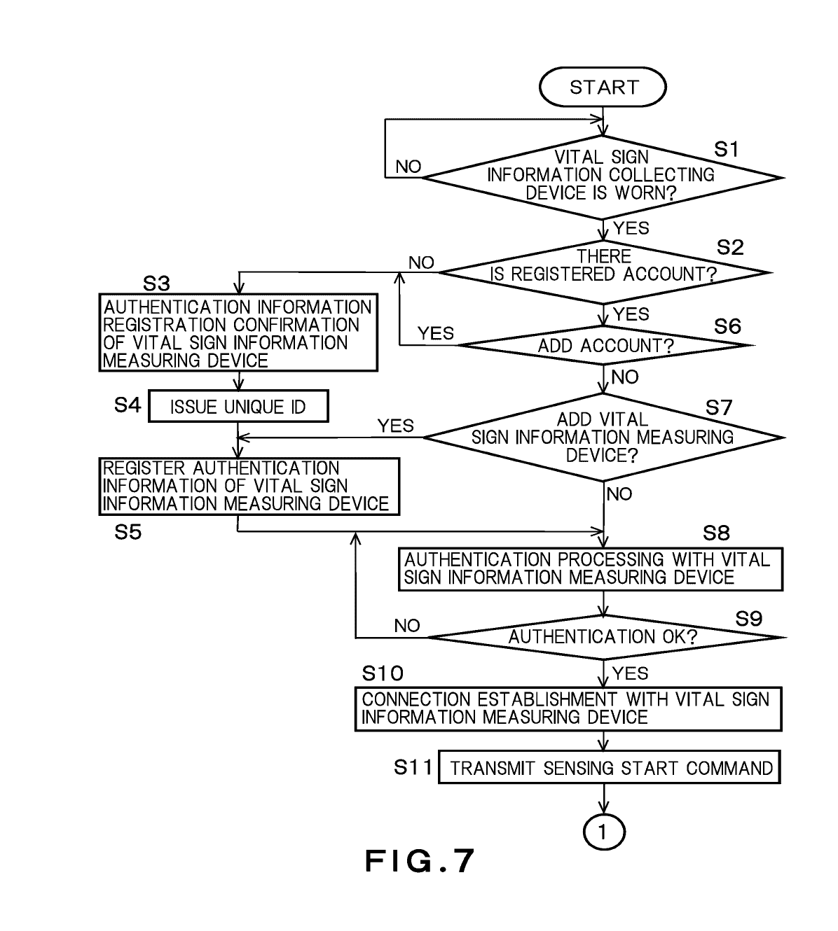

Here, the pairing of the vital sign information measuring device 2 and the vital sign information collecting device 3 is described with reference to a flowchart of FIG. 7. First, it is determined whether or not the user wears the vital sign information collecting device 3 (step S1). Regarding the determination of whether or not the user wears the device, wearing/non-wearing can be easily determined, if, for example, a switch is provided to a wearing portion of the vital sign information collecting device 3 and the switch is turned on when the device is worn. However, wearing determination is not essential, and a wearing state may be notified by user operation.

The processing stays in step S1 until the vital sign information collecting device 3 is worn, and when the device is worn, it is determined whether or not there is a registered account of the vital sign information measuring device 2 (step S2). When there is no account, it is inquired to the user whether or not the authentication information of the vital sign information measuring device 2 is registered (step S3), and a unique ID is issued to the measurement subject in a case in which the user desires (step S4), and then the authentication information of the vital sign information measuring device 2 is registered (step S5).

On the other hand, when it is determined that there is a registered account in step S2, it is determined whether or not an account is added (step S6). In a case in which the account is added, the processing proceeds to step S3 described above, and in a case in which the account is not added, it is determined whether or not the vital sign information measuring device 2 that is used is added (step S7), and in the case in which the account is added, the processing proceeds to step S5.

In a case in which it is determined that the account is not added in step S7, or the processing of step S5 is ended, authentication processing with the vital sign information measuring device 2 is performed (step S8). Next, it is determined whether or not the authentication is OK (step S9), and the processing returns to step S8 when the authentication is not OK, and connection is established with the vital sign information measuring device 2 when the authentication is OK (step S10). The connection of this case is, for example, connection of Bluetooth (registered trademark). When the connection is established, a sensing start command is transmitted to the vital sign information measuring device 2 (step S11). Thus, the vital sign information measuring device 2 starts sensing, and stores the vital sign information measured in the vital sign information measuring device 2. The information stored is automatically transmitted to the vital sign information collecting device 3 after a certain time elapses. Alternatively, the vital sign information measuring device 2 can be configured to successively transmit the vital sign information measured after the sensing start, to the vital sign information collecting device 3.

### Second Embodiment

The vital sign information collecting device 3 may include a function of measuring a state of the measurement subject, besides the function of collecting the vital sign information measured by the vital sign information measuring device 2. In this case, the vital sign information collecting device 3 can be used to continuously measure the vital sign information with the vital sign information measuring device 2 while a person wears the vital sign information measuring device 2, and, in a case in which the person takes off the vital sign information measuring device 2, instead, the vital sign information collecting device 3 continuously measures the state of the measurement subject. Thus, as long as the person wears the vital sign information collecting device 3, regardless of whether or not the person wears the vital sign information measuring device 2, it is possible to always continue to measure any vital sign information, and to precisely record the person's sleep, behavior, or the like.

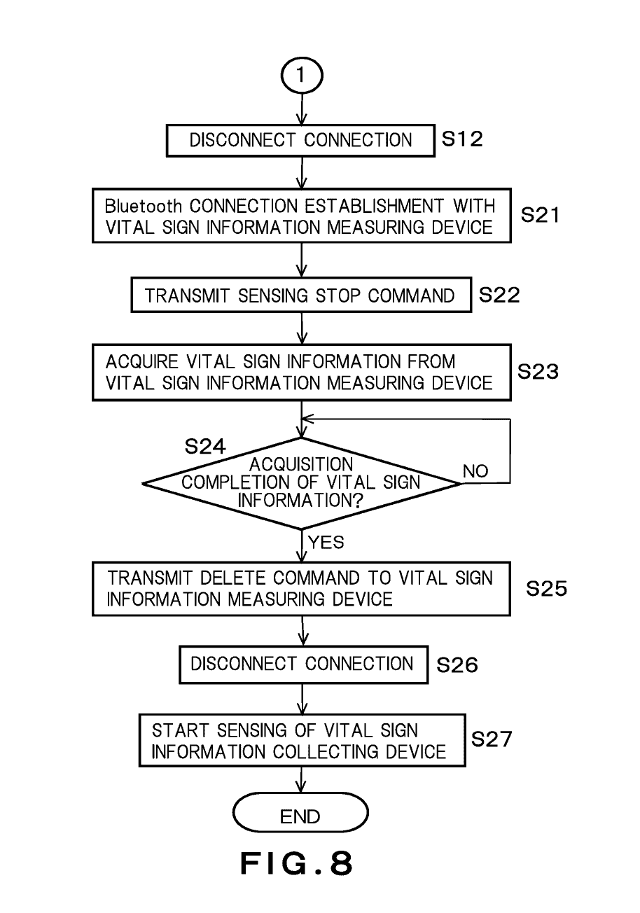

In a second embodiment, an aspect is described in which the vital sign information measuring device 2 and the vital sign information collecting device 3 work together, thereby seamlessly executing the various types of sensing for the measurement subject. FIG. 8 is a flowchart illustrating an example of a processing procedure of the vital sign information collecting device 3 of a case in which the vital sign information measuring device 2 ends sensing operation and then the vital sign information collecting device 3 starts sensing operation.

In this embodiment, the vital sign information collecting device 3 that has transmitted the sensing start command in step S11 of FIG. 7 disconnects the connection with the vital sign information measuring device 2 (step S12 of FIG. 8). After that, the vital sign information measuring device 2 continues measurement, and retains the data in a storage area of the vital sign information measuring device 2. Alternatively, it may be made so that the vital sign information measuring device 2 continues measurement without disconnecting the connection, and the vital sign information collecting device 3 successively receives measurement information.

First, connection with the vital sign information measuring device 2 is established of, for example, Bluetooth (step S21). When the connection is established, a sensing stop command is transmitted to the vital sign information measuring device 2 (step S22). When the vital sign information measuring device 2 that has received the command stops sensing operation and transmits the vital sign information stored in the data storage unit 43, the vital sign information is acquired (step S23). Next, it is determined whether or not acquisition of the vital sign information is completed (step S24). Step S23 and S24 are repeated until acquisition is completed, and when the acquisition is completed, a delete command is transmitted to the vital sign information measuring device 2 (step S25). The vital sign information measuring device 2 that receives the command deletes the transmitted vital sign information. After that, the connection with the vital sign information measuring device 2 is disconnected (step S26), and after that, sensing operation is started by a sensor incorporated in the vital sign information collecting device 3 (step S27).

Regarding the processing of the present embodiment, the following usage is considered, for example. Before the user goes to bed, step S12 (or a series of flow including previous steps) is performed, and while the user is sleeping, the vital sign information measuring device 2 continuously measures the vital sign information about a sleep state of the user, and the vital sign information measured is stored in the vital sign information measuring device 2. When the user wakes up in the morning, the processing of steps S21 to S27 is performed. At this time, the vital sign information stored in the vital sign information measuring device 2 is transmitted to the vital sign information collecting device 3, and, after that, the vital sign information collecting device 3 can start measurement using the sensor in the vital sign information collecting device 3, of, for example, an amount of activity or behavior of the user, in which the object or quality is different from the information measured by the vital sign information measuring device 2 until then.

In this way, with the processing procedure of FIG. 8, the vital sign information measuring device 2 and the vital sign information collecting device 3 can continuously acquire the vital sign information of the user by sharing.

Incidentally, in the above description, the vital sign information measuring device 2 is stopped before the measurement of the vital sign information collecting device 3 is started; however, it is possible to simultaneously perform vital sign information measurement by the vital sign information measuring device 2 and measurement (behavior measurement) of a state of a wearer by the vital sign information collecting device 3. In this way, the vital sign information measured by the vital sign information measuring device 2 and the behavior information measured by the vital sign information collecting device 3 can be compared, and a detailed analysis of the vital sign information becomes possible by looking back what behavior has been done at that time, about the vital sign information measured.

Regarding the behavior information, it may be made to mount an audio recording (or voice recognition) function on the vital sign information collecting device 3, and to allow the behavior information to be input by voice.

### Third Embodiment

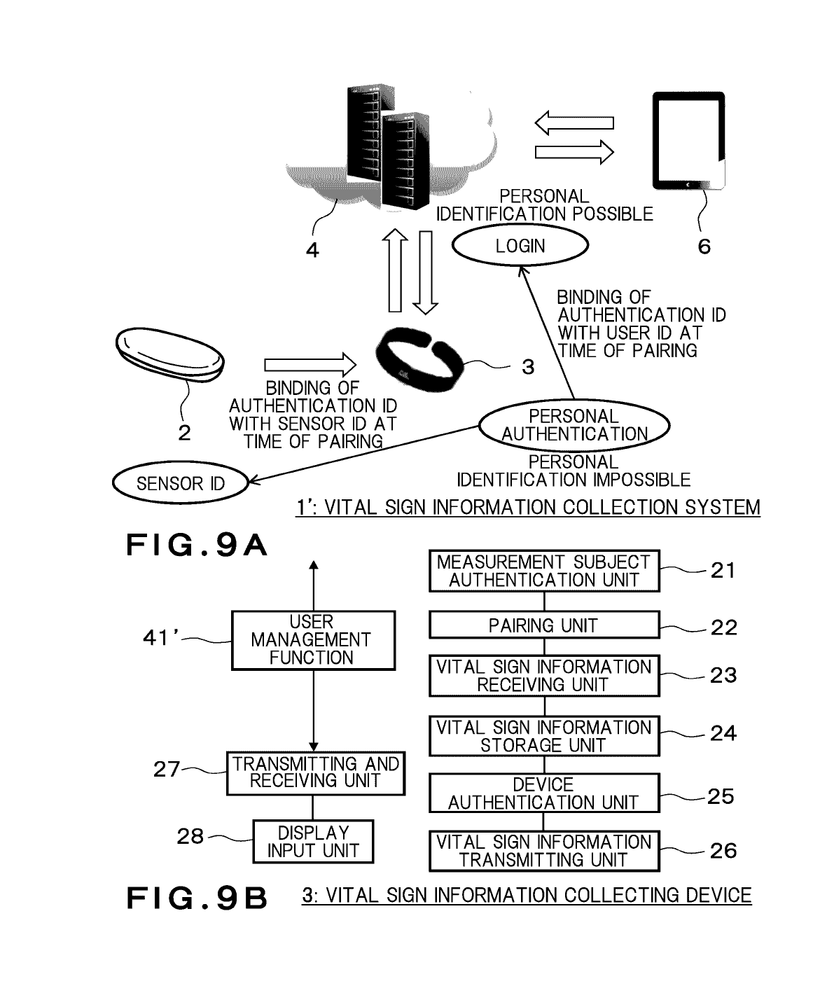

FIG. 9A is a configuration diagram of a vital sign information collection system according to a third embodiment of the present invention. In the present embodiment, the authentication ID and the user ID are linked with each other in the server 4 without using the information terminals 5, 5′.

Here, the vital sign information collecting device 3 includes a transmitting and receiving unit 27 that transmits and receives a file and the like to/from the server 4, and a display input unit 28, besides the components described in the first embodiment (FIG. 9B). The transmitting and receiving unit 27 transmits the user ID capable of substantially uniquely identifying an individual input in the display input unit 28, to the server 4 by using the user management function 41′ prepared in the server 4. The server 4 manages the user ID that has been transmitted to the server, and the authentication ID and vital sign information that have been separately transmitted to the server linked with each other. Such server 4 is a management device that retains the vital sign information linked with the personal information capable of identifying the measurement subject.

### Fourth Embodiment

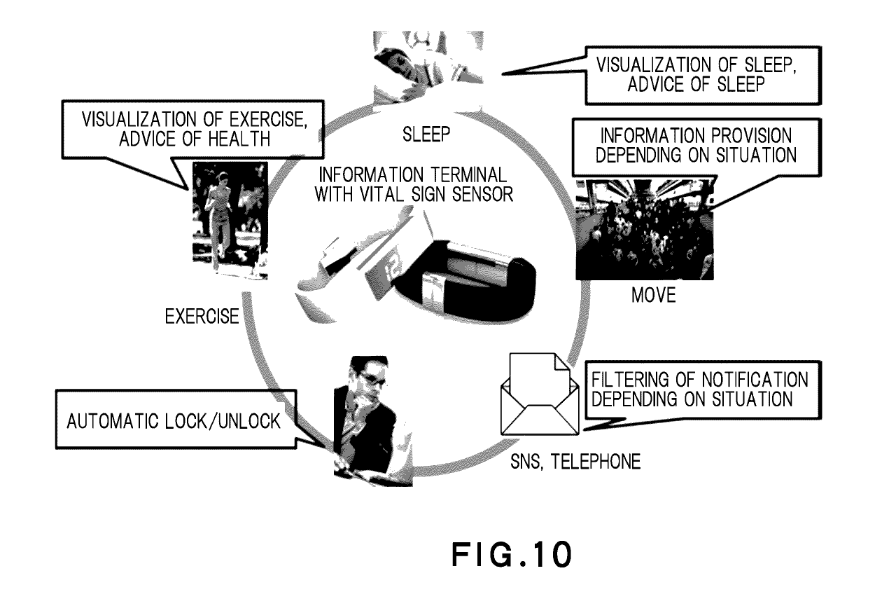

FIG. 10 is a diagram conceptually describing functions of a vital sign information collecting device 3 with a biological sensor that measures vital sign information. The vital sign information collecting device 3 of FIG. 10 includes, for example, at least one of an automatic lock/unlock function of the information terminal 5, a filtering function of notification of an e-mail, telephone, or the like; an information provision function, a sleep analysis function; and an exercise analysis function.

The automatic lock/unlock function of the information terminal 5 can be achieved by providing a short-range wireless communication function to the vital sign information collecting device 3, besides the authentication function. That is, the vital sign information collecting device 3 transmits the authentication information of the authentication in the vital sign information collecting device 3 to the information terminal 5 through the short-range wireless communication. The information terminal 5, when the authentication information is transmitted from the vital sign information collecting device 3, determines that it is an authorized user, and allows use of the information terminal 5, that is, automatically unlocks the information terminal 5. When a person who wears the vital sign information collecting device moves to a location in which the short-range wireless communication with the information terminal 5 is impossible, use of the information terminal 5 is prohibited, that is, the information terminal 5 is automatically locked.

The filtering function automatically stops a telephone or e-mail ring tone, in a case in which it is determined that the user is currently sleeping, from the vital sign information acquire by the vital sign information collecting device 3, for example.

Alternatively, it can be made so that, when it is difficult to respond all of the telephone and e-mail due to a user's state, such as running, a caller of the telephone or a sender of the e-mail and/or a title are only notified in the first stage, and in a case in which the user determines to respond the telephone or to check the contents of the e-mail, response to the telephone or check of the e-mail is performed in the second stage.

The information provision function displays information such as the number of steps, on a display screen of the vital sign information collecting device 3, in a case in which it is determined that the user is moving, from the vital sign information acquired by the vital sign information collecting device 3, for example. In a case in which it is on the way home, and a target value of the number of steps of the day has not achieved, and it is on the train and there is a time margin (+ weather is fine), when the train approaches a station one stop before a station normally used, because the target number of steps has not achieved, it is proposed to get off the train at the station one stop before, to walk. If there is bargain sale information or the like of a shop on the way home, the information is also notified to encourage the user to walk. In this way, by performing information provision depending on the situation, it becomes possible to effectively prompt lifestyle improvement.

The sleep analysis function displays information about the sleep state of the user or advice about sleep on the display screen of the vital sign information collecting device 3, according to the vital sign information acquired by the vital sign information collecting device 3, for example.

The exercise analysis function, for example, detects the amount of activity of the user from the vital sign information acquired by the vital sign information collecting device 3, and displays the amount of activity and advice about exercise on the display screen of the vital sign information collecting device 3.

In this way, in the present embodiment, the vital sign information measured by the vital sign information measuring device 2 is collected by the vital sign information collecting device 3, and then the vital sign information is acquired by the management device, and the vital sign information is managed linked with the personal information, so that various types of vital sign information for each user can be centrally managed by the management device. Therefore, the vital sign information can be analyzed in detail for each type of the vital sign information, and on the basis of the analysis result of the vital sign information for each user, information about sleep, exercise, and health can be provided to each user.

At least a part of the vital sign information collection system 1 described in the above embodiments may be configured by hardware, and may be configured by software. In a case in which it is configured by the software, a program that realizes at least a part of the function of the vital sign information collection system 1 may be stored in a recording medium such as a flexible disk or CD-ROM, and may be executed by a computer that reads the program. The recording medium is not limited to a detachable medium such as a magnetic disk or optical disk, and may be a fixed type recording medium such as a hard disk device or memory.

In addition, the program that realizes at least a part of the function of the vital sign information collection system 1 may be distributed via a communication line (including wireless communication) such as the Internet. Further, the program may be distributed in a state of being encrypted, modulated, or compressed, via a wired line or wireless line such as the Internet, or stored in the recording medium.

Although several embodiments of the present invention have been described, these embodiments have been presented by way of examples, and are not intended to limit the scope of the invention. These novel embodiments can be embodied in various other forms, and various omissions, substitutions, modifications can be made without departing from the scope of the invention. These embodiments and their modifications are included within the scope and spirit of the invention, and included within the scope of the invention as defined by the appended claims and equivalents thereof.

1. A vital sign information collection system comprising:
a vital sign information measuring device that measures vital sign information;
a vital sign information collecting device that performs authentication by attribute information of a measurement subject and collects the vital sign information measured by the vital sign information measuring device, and links the attribute information with the collected vital sign information; and
a management device that acquires the attribute information and the vital sign information linked with each other, from the vital sign information collecting device, and retains the vital sign information linked with personal information to identify the measurement subject.

2. The vital sign information collection system according to claim 1, wherein
the management device is an information terminal, and has a capability of transmitting the retained vital sign information and the personal information to a server.

3. The vital sign information collection system according to claim 1, wherein
the management device is a server, and the server inquires personal information to identify the measurement subject of an information terminal or the vital sign information collecting device, and receives the personal information to identify the measurement subject from the information terminal or the vital sign information collecting device.

4. The vital sign information collection system according to claim 1, wherein
the vital sign information measuring device comprises at least one of a device that measures unique vital sign information of the measurement subject, and a device that measures vital sign information of many and unspecified persons.

5. The vital sign information collection system according to claim 1, wherein
the vital sign information measuring device measures at least one of an electrocardiogram, a pulse wave, an acceleration, and a temperature.

6. The vital sign information collection system according to claim 1, wherein
the vital sign information collecting device has a capability of being worn on a body of a measurement subject to which authentication has been performed, and collects vital sign information from the vital sign information measuring device while being worn on the body of the measurement subject.

7. The vital sign information collection system according to claim 6, wherein
the vital sign information collecting device invalidates information to which authentication has been performed, when taken off from the body of the measurement subject to which authentication has been performed.

8. The vital sign information collection system according to claim 7, wherein
the information terminal validates authentication information to which authentication has been performed by the authentication device while the vital sign information collecting device is worn on the body of the measurement subject, and invalidates the authentication information when the vital sign information collecting device is taken off from the body of the measurement subject.

9. The vital sign information collection system according to claim 1, wherein
the management device transmits acquisition completion information indicating that acquisition of the vital sign information collected by the vital sign information collecting device is completed, to the vital sign information collecting device, and
the vital sign information collecting device receives the acquisition completion information and then deletes the vital sign information acquired by the management device.

10. The vital sign information collection system according to claim 1, wherein
the vital sign information collecting device interprets the vital sign information measured by the vital sign information measuring device, and, on the basis of the interpreted vital sign information, variably adjusts information granularity of the vital sign information that is transmitted to the management device.
